(19) United States
(12) Patent Application Publication
(10) Pub. No.: US 2018/0117142 A1
(43) Pub. Date: May 3, 2018

(54) METHOD OF TREATING PATIENTS WITH NON-IgE MEDIATED HYPERSENSITIVITIES TO ENVIRONMENTAL ALLERGENS

(71) Applicant: Beaufort County Allergy, LLC, Hilton Head Island, SC (US)

(72) Inventor: Thomas Charles Beller, Hilton Head Island, SC (US)

(73) Assignee: Beaufort County Allergy, LLC, Hilton Head Island, SC (US)

(21) Appl. No.: 15/718,157

(22) Filed: Sep. 28, 2017

Related U.S. Application Data
(60) Provisional application No. 62/415,720, filed on Nov. 1, 2016.

(51) Int. Cl. A61K 39/35; A61K 9/00; A61K 49/00

(52) U.S. Cl. CPC A61K 39/35; A61K 9/0019; A61K 2039/54; A61K 49/0006; A61K 9/006

(57) ABSTRACT

Methods and systems for treating delayed type hypersensitivity to allergens as well as treating various disorders associated with same.

### BACKGROUND OF THE INVENTION

### 1) Field of the Invention

The present invention relates to a method of treating patients with non-IgE mediated hypersensitivities to environmental allergens including delayed type hypersensitivity (DTH).

### 2) Description of Related Art

In the context of improved attention to clean food, clean water, and waste removal, industrialized countries have limited their exposure to microbes, parasites, and disease-causing bacteria. Concomitantly, there has been a significant increase in the incidence of chronic inflammatory disorders (e.g., allergic and autoimmune disorders). Several epidemiologic studies have described an evolutionary link or “evolved dependence” between the increased incidence of allergy and the more limited exposure to microbes, parasites, and other “old friends.” Rather than provoking aggressive immune responses, these no-longer-present organisms have been found to be associated with a pattern of maturation of dendritic cells and regulatory T cells helping to control mediators of inflammation.

Pathogen-associated molecular patterns, or PAMPs, are molecules associated with groups of pathogens, that are recognized by cells of the innate immune system. These molecules can be referred to as small molecular motifs conserved within a class of microbes. They are recognized by toll-like receptors (TLRs) and other pattern recognition receptors (PRRs) in both plants and animals. A vast array of different types of molecules can serve as PAMPs, including glycans and glycoconjugates.

PAMPs activate innate immune responses, protecting the host from infection, by identifying some conserved non-self-molecules. Bacterial lipopolysaccharides (LPSs), endotoxins found on the cell membranes of gram-negative bacteria, are considered to be the prototypical class of PAMPs. LPSs are specifically recognized by TLR4, a recognition receptor of the innate immune system. Other PAMPs include bacterial flagellin (recognized by TLR5), lipoteichoic acid from gram-positive bacteria, peptidoglycan, and nucleic acid variants normally associated with viruses, such as double-stranded RNA (dsRNA), recognized by TLR3 or unmethylated CpG motifs, recognized by TLR9. Although the term “PAMP” is relatively new, the concept that molecules derived from microbes must be detected by receptors from multicellular organisms has been held for many decades, and references to an “endotoxin receptor” are found in much of the older literature.

The word “allergy” was first used by Clemens von Pirquet in 1906. Allergies, also known as allergic diseases, are a number of conditions caused by hypersensitivity of the immune system to something in the environment that usually causes little or no problem in most people. These diseases include hay fever, food allergies, atopic dermatitis, allergic asthma, and anaphylaxis. Symptoms may include red eyes, an itchy rash, sneezing, a runny nose, shortness of breath, or swelling. Food intolerances and food poisoning are separate conditions.

Common allergens include pollen, dust mites, pet dander, mold spores and certain foods. Food, insect stings, and medications are common causes of severe reactions. Their development is due to both genetic and environmental factors. The underlying mechanism often involves immunoglobulin E antibodies (IgE), part of the body's immune system, binding to an allergen and then to a receptor on mast cells or basophils where it triggers the release of inflammatory chemicals such as histamine. IgE antibodies are thought to be an essential component of all allergic responses to environmental allergens causing allergic rhinitis, allergic conjunctivitis and allergic asthma. Diagnosis is typically based on a person's medical history and allergy tests that look specifically for IgE antibodies targeted against varying allergens. Positive tests may not mean there is a significant allergy to the substance in question since sensitization and the presence of IgE antibodies does not always lead to clinical reactions when exposed to the allergen. However, the absence of IgE antibodies rules out a diagnosis of allergic rhinitis, allergic conjunctivitis and allergic asthma according to today's standards and the present understanding of allergic pathways.

Early exposure to potential allergens may be protective. Treatments for allergies include avoiding known allergens and the use of medications such as steroids and antihistamines. In severe reactions injectable adrenaline (epinephrine) is recommended. Allergen immunotherapy, which gradually exposes people to larger and larger amounts of allergen, is known to be useful for environmental allergies causing rhinoconjunctivitis or asthma and for insect stings causing anaphylaxis. Its use in IgE-mediated food allergies is emerging.

Allergies are common. In the developed world, about 20% of people are affected by allergic rhinitis, about 6% of people have at least one food allergy, and about 20% have atopic dermatitis at some point in time. Depending on the country about 1-18% of people have asthma. Anaphylaxis occurs in between 0.05-2% of people. Rates of many allergic diseases appear to be increasing.

Allergic rhinoconjunctivitis and allergic asthma have been defined by the presence of IgE antibodies in the setting of typical clinical symptoms. Allergy tests look specifically for these antibodies, typically in one of two ways: (1) skin tests look for histamine release caused by cross-bridging of IgE while (2) blood tests look for IgE through tests called Radio Allergo Sorbent Tests (RAST) or a newer test called ImmunoCap (sometimes called CapRAST). If IgE antibodies are not identified a patient with rhinoconjunctivitis or asthma symptoms would be said to have “non-allergic” rhinoconjunctivitis or “non-allergic” asthma. However, certain pathways involving hypersensitivity responses to environmental allergens that lead to allergy symptoms apparently do not involve IgE antibodies.

Accordingly, it is an object of the present invention to provide improved methods for treating various maladies via treatment of non-IgE mediated hypersensitivity responses to environmental allergens.

### SUMMARY OF THE INVENTION

The above objectives are accomplished according to the present invention by providing in a first embodiment, a method for treating non-IgE mediated hypersensitivities to allergens. The method includes providing immunotherapy to a mammal exhibiting allergy symptoms via administering allergens to a mammal via a delivery system, wherein the mammal has negative test results for IgE antibodies but who exhibits delayed type hypersensitivity to one or more allergens. Further, the delivery system for the allergens includes subcutaneous injections, liquid sublingual drops, tablets, or combinations of the above. Still further, subcutaneous injections may be delivered over increasing time intervals. Further yet, the dose of allergen may remain substantially constant during administration of the allergen. Yet still further, the dose of allergen administered via subcutaneous injections or via liquid sublingual drops may be 1,000 to 1,000,000 times lower than allergen doses for IgE mediated allergy responses. Further yet, the method of claim 1 may be used to treat acid reflux, atypical acid reflux (neurophylaxis), non-allergic rhinoconjunctivitis, non-allergic asthma and inflammatory conditions such as Crohn's disease, Ulcerative Colitis, Systemic Lupus Erythematosus, Rheumatoid Arthritis, Kawasaki's vasculitis, Ankylosing spondylitis, Idiopathic Pulmonary Fibrosis, Alzheimer's disease, and/or Coronary Artery disease. Further still, the allergens employed with the method may be selected from the list comprising Dermatophagoides farina, Dermatophagoides pteronyssinus, Dust mite mix, cockroach, dog extract, cat extract, horse extract, Ragweed extract, cocklebur, English plaintain, marsh elder, lamb's quarter, pigweed, mugwort, Japanese hop, Bermuda, timothy, meadow fescue, June, Orchard, Red Top, Perennial Rye, Bahia, Sweet Vernal, Brome, Johnson, White ash, privet, White birch, Alder, Oak, Beech, Cottonwood, Black willow, poplar, pecan, black walnut, shagbar hickory, maple, box elder, elm, sweetgum, Sycamor, cedar, bayberry, mulberry, Alternaria, Aspergillus, Helminthosporium, Hormodendrum, Penicillium, Fusarium, Mucor, Phoma, Candida, Epicoccum, Rhizopus, Pullularia, Curvularia, Stemphyllium and Chaetonium and/or any combinations of the above. Furthermore any pollens, molds or other animal extracts not listed could also be used.

In an alternative embodiment, a method for treating allergic reactions is provided. The method includes treating allergic reactions in a mammal that has negative tests for IgE and who may or may not exhibit delayed type hypersensitivity to any allergens. In this embodiment, the treatment is aimed at reducing Toll Like Receptor activation from environmental allergens. At least one allergen causing an allergic reaction in the mammal possesses a pathogen associated molecular pattern similar to a parasitic pathogen associated molecular pattern, Immunotherapy is provided to the mammal via administering allergens to the mammal via a delivery system Further, the delivery system for the allergens includes subcutaneous injections, liquid sublingual drops, tablets, or combinations of the above. Still further, subcutaneous injections may be delivered over increasing time intervals. Further yet, the dose of allergen may remain substantially constant during administration of the allergen. Yet still further, the dose of allergen administered via subcutaneous injections or via liquid sublingual drops may be 1,000 to 1,000,000 times lower than allergen doses for IgE mediated allergy responses. Still yet further, the method may be used to treat acid reflux, atypical acid reflux (neurophylaxis), non-allergic rhinoconjunctivitis, non-allergic asthma and inflammatory conditions such as Crohn's disease, Ulcerative Colitis, Systemic Lupus Erythematosus, Rheumatoid Arthritis, Kawasaki's vasculitis, Ankylosing spondylitis, Idiopathic Pulmonary Fibrosis, Alzheimer's disease, and/or Coronary Artery disease. Further still, the allergens employed with the method may be selected from the list comprising Dermatophagoides farina, Dermatophagoides pteronyssinus, Dust mite mix, cockroach, dog extract, cat extract, horse extract, Ragweed extract, cocklebur, English plaintain, marsh elder, lamb's quarter, pigweed, mugwort, Japanese hop, Bermuda, timothy, meadow fescue, June, Orchard, Red Top, Perennial Rye, Bahia, Sweet Vernal, Brome, Johnson, White ash, privet, White birch, Alder, Oak, Beech, Cottonwood, Black willow, poplar, pecan, black walnut, shagbar hickory, maple, box elder, elm, sweetgum, Sycamor, cedar, bayberry, mulberry, Alternaria, Aspergillus, Helminthosporium, Hormodendrum, Penicillium, Fusarium, Mucor, Phoma, Candida, Epicoccum, Rhizopus, Pullularia, Curvularia, Stemphyllium and Chaetonium and/or any combinations of the above. Furthermore any pollens, molds or other animal extracts not listed could also be used.

In a further alternative embodiment, a method for treating gastroesophageal reflux disease in a mammal is provided. The method includes administering immunotherapy to a mammal, who may or may not have IgE antibody responses and who may or may not exhibit delayed type hypersensitivity, via a delivery system which contains allergens. Further, the delivery system for the allergen may comprise subcutaneous injections, liquid sublingual drops, tablets, or combinations of the above. Still further, the subcutaneous injections may be delivered over increasing time intervals. Further yet, the dose of allergen provided by the delivery system may remain substantially constant during the administration of the allergen. Furthermore, a dose of allergen administered via subcutaneous injections or liquid sublingual drops may be 1,000 to 1,000,000 times lower than an allergen dose for IgE mediated allergy responses. Yet still further, the allergens employed with the method may be selected from the list comprising Dermatophagoides farina, Dermatophagoides pteronyssinus, Dust mite mix, cockroach, dog extract, cat extract, horse extract, Ragweed extract, cocklebur, English plaintain, marsh elder, lamb's quarter, pigweed, mugwort, Japanese hop, Bermuda, timothy, meadow fescue, June, Orchard, Red Top, Perennial Rye, Bahia, Sweet Vernal, Brome, Johnson, White ash, privet, White birch, Alder, Oak, Beech, Cottonwood, Black willow, poplar, pecan, black walnut, shagbar hickory, maple, box elder, elm, sweetgum, Sycamor, cedar, bayberry, mulberry, Alternaria, Aspergillus, Helminthosporium, Hormodendrum, Penicillium, Fusarium, Mucor, Phoma, Candida, Epicoccum, Rhizopus, Pullularia, Curvularia, Stemphyllium and Chaetonium and/or any combinations of the above. Furthermore any pollens, molds or other animal extracts not listed could also be used.

In a further alternative embodiment, a method for treating neurophylaxis in a mammal is provided. Neurophylaxis is an expanded view of atypical acid reflux that recognizes the nervous system, in response to stomach acid, participates in the development of diffuse symptoms such as headache, itchy eyes, watery eyes, sneezing, runny nose, nasal congestion, post nasal drip, sinus pressure, cough, hoarse voice, throat clearing, shortness of breath, chest tightness, itchy skin, urinary urgency, vaginal irritation, itchy anus and irritable bowel (with diarrhea and/or constipation). The method includes administering immunotherapy to a mammal via a delivery system which contains allergens. Further, the delivery system for the allergen may comprise subcutaneous injections, liquid sublingual drops, tablets, or combinations of the above. Still further, the subcutaneous injections may be delivered over increasing time intervals. Further yet, the dose of allergen provided by the delivery system may remain substantially constant during the administration of the allergen. Furthermore, a dose of allergen administered via subcutaneous injections or liquid sublingual drops may be 1,000 to 1,000,000 times lower than an allergen dose for IgE mediated allergy responses. Yet still further, the allergens employed with the method may be selected from the list comprising Dermatophagoides farina, Dermatophagoides pteronyssinus, Dust mite mix, cockroach, dog extract, cat extract, horse extract, Ragweed extract, cocklebur, English plaintain, marsh elder, lamb's quarter, pigweed, mugwort, Japanese hop, Bermuda, timothy, meadow fescue, June, Orchard, Red Top, Perennial Rye, Bahia, Sweet Vernal, Brome, Johnson, White ash, privet, White birch, Alder, Oak, Beech, Cottonwood, Black willow, poplar, pecan, black walnut, shagbar hickory, maple, box elder, elm, sweetgum, Sycamor, cedar, bayberry, mulberry, Alternaria, Aspergillus, Helminthosporium, Hormodendrum, Penicillium, Fusarium, Mucor, Phoma, Candida, Epicoccum, Rhizopus, Pullularia, Curvularia, Stemphyllium and Chaetonium and/or any combinations of the above. Furthermore any pollens, molds or other animal extracts not listed could also be used.

In a still further embodiment, a delayed type hypersensitivity test is provided. The test includes injecting a mammal with a reduced concentration of allergen stock, monitoring the mammal for signs of a reaction to the allergen stock, wherein a reaction to the allergen stock is delayed in that no immediate reaction to the allergen stock occurs but instead an initial reaction begins hours after the injection and peaks hours after the initial reaction. A delayed type hypersensitivity test is useful to identify that a delayed, non-IgE mediated allergy response is active in the mammal being tested. It also identifies that the immune system is fighting a specific allergen and that other underlying immune responses may be active even if that specific immune response is not involved in the development of a positive DTH test. For example, a positive DTH test may identify an increased likelihood that a Toll Like Receptor response may be active in the mammal reacting to the test.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows an image of the stomach with a close-up view of histamine and the histamine type 2 (H2) receptor activating the proton pump that makes acid.

FIG. 2 shows an illustration of the classic IgE defensive response against parasites.

FIG. 3 shows representative images of parasites and allergens.

FIG. 4 shows a representative image of acid reflux.

FIG. 5 illustrates neurophylaxis.

FIG. 6 shows a picture of an arm that was injected with a high dose of tree pollens.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Broadly, an embodiment of the present invention provides a method of treatment comprising: immunotherapy in the form of allergy shots, liquid sublingual drops or tablets containing allergens administered to patients having negative traditional allergy tests for IgE antibodies. For example, allergy extracts such as those sold by Stellergenes Greer, Jubilant Hollister Stier or ALK Abello can be used for subcutaneous injections or sublingual treatments, and tablets such as Grastek™, a tablet for grass allergy, or Ragwitek™, a tablet for ragweed allergy, may be taken to reduce DTH. The ingredients in these allergy extracts is the same allergens that occur naturally in the environment and that cause symptoms of hay fever, asthma and other allergic conditions. This therapy could reduce delayed type hypersensitivity and could reduce other immune pathways that remain difficult to identify, for example, pathways involving Toll Like Receptors (TLRs).

As mentioned above, certain pathways involved in the development of allergy symptoms do not involve IgE antibodies. Some of these pathways start in the stomach and are activated by stomach acid. Stomach acid then activates nerves which release an irritating neuromediator (probably substance P) at distant sites to create expelling, flushing and defensive responses. For example, sneezing, coughing, runny nose, watery eyes are obvious expelling and flushing responses.

Substance P (SP) is an undecapeptide (a peptide composed of a chain of 11 amino acid residues) member of the tachykinin neuropeptide family. It is a neuropeptide, acting as a neurotransmitter and as a neuromodulator. Substance P and its closely related neurokinin A (NKA) are produced from a polyprotein precursor after differential splicing of the preprotachykinin A gene. The deduced amino acid sequence of substance P is as follows: Arg Pro Lys Pro Gln Phe Gly Leu Met (RPKPQQFFGLM) with an amidation at the C-terminus. Substance P is released from the terminals of specific sensory nerves. It is found in the brain and spinal cord and is associated with inflammatory processes and pain. Substance P is a small peptide that is released when your nervous system is stimulated. It is involved in regulation of the pain threshold (the point at which sensation is perceived as pain.) Increased levels of substance P can make nerves more sensitive to pain and heighten your awareness of pain.

The endogenous receptor for substance P is neurokinin 1 receptor (NK1-receptor, NK1R). It belongs to the tachykinin receptor sub-family of GPCRs. Other neurokinin subtypes and neurokinin receptors that interact with SP have been reported as well. Amino acid residues that are responsible for the binding of SP and its antagonists are present in the extracellular loops and transmembrane regions of NK-1. Binding of SP to NK-1 results in internalization by the clathrin-dependent mechanism to the acidified endosomes where the complex disassociates. Subsequently, SP is degraded and NK-1 is re-expressed on the cell surface. Substance P and the NK1 receptor are widely distributed in the brain and are found in brain regions that are specific to regulating emotion (hypothalamus, amygdala, and the periaqueductal gray). They are found in close association with serotonin (5-HT) and neurons containing norepinephrine that are targeted by the currently used antidepressant drugs. The SP receptor promoter contains regions that are sensitive to cAMP, AP-1, AP-4, CEBPB, and epidermal growth factor. Because these regions are related to complexed signal transduction pathways mediated by cytokines, it has been proposed that cytokines and neurotropic factors can induce NK-1. Also, SP can induce the cytokines that are capable of inducing NK-1 transcription factors.

The “P” in substance “P” [SP] is mistakenly thought to signify Pain or Psychiatric substance. Substance P (“P” standing for “Preparation” or “Powder”) is a neuropeptide—but only nominally so, as it is ubiquitous. Its receptor—the neurokinin type 1—is distributed over cytoplasmic and nuclear membranes of many cell types (neurons, glia, endothelia of capillaries and lymphatics, fibroblasts, stem cells, white blood cells) in many tissues and organs. SP amplifies or excites most cellular processes.

Substance P is a key first responder to most noxious/extreme stimuli (stressors), i.e., those with a potential to compromise biological integrity. SP is thus regarded as an immediate defense, stress, repair, survival system. The molecule, which is rapidly inactivated (or at times further activated by peptidases) is rapidly released, repetitively and chronically, as warranted, in the presence of a stressor. Unique among biological processes, SP release (and expression of its NK1 Receptor (through autocrine, paracrine, and endocrine-like processes)) may not naturally subside in diseases marked by chronic inflammation (including cancer). The SP or its NK1R, as well as similar neuropeptides, appear to be vital targets capable of satisfying many unmet medical needs.

The applicant has found that these symptoms can be blocked with medications that reduce stomach acid production, such as Prilosec™ and by medications, such as Gabapentin™ and Baclofen™ that slow nerve activity. Gabapentin™ and Baclofen™ have both been shown to reduce the release of the neuromediator, substance P. Allergens activate these pathways by stimulating Toll Like Receptors (TLRs) which are expressed on nerves and are associated with amplified nociception. TLRs are activated by Pathogen Associated Molecular Patterns (PAMPs) on parasites and the pathway described above is thought to have a purpose in helping to defend from invasion by parasites.

Stomach acid has been the human body's first line of defense against parasites for millions of years. Over time the body learned to use stomach acid as a surrogate signal for the presence of parasites. There is evidence for this since patients with acid reflux tend to have parasite killing cells, called Eosinophils, infiltrating the esophagus. Allergens have also been found to have PAMPs which activate this pathway. The pathway may be called “neurophylaxis” (phylaxis is to fend off and neuro implies that this response comes from nerves) or “allergic neurophylaxis.”

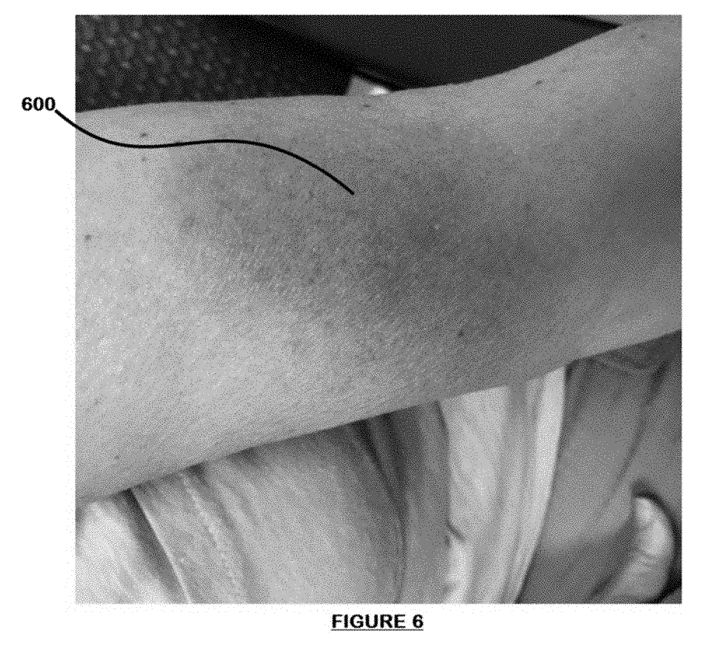

Neurophylaxis would have been a normal response in the presence of parasites. For example, if someone were swimming in a pond and drinking parasite infested water, it would be beneficial for stomach acid to stimulate expelling, flushing, and other defensive responses at potential points of exposure. When this pathway is overactive in the setting of allergic exposure, this may be called “allergic neurophylaxis.” Other symptoms can include urinary frequency/urgency, diarrhea and/or constipation (often called irritable bowel), sinus pressure, headaches, tinnitus, vertigo, bedwetting, and more (see FIG. 6 in the figure irritable bowel is not mentioned because this is a new discovery). Activation of this pathway can occur through contact with allergens whether IgE antibodies are present or not. This means that patients who have been labelled as having non-allergic rhinoconjunctivitis or non-allergic asthma may actually have stimulation of their symptoms from allergens.

FIG. 6 shows a photograph of an arm that was injected with a high dose of tree pollens. The reaction area 600 started to occur at 8 hours and peaked in intensity at 36 hours representing a delayed type hypersensitivity (DTH) response. The patient in this picture had negative RAST tests as well as negative skin prick and intradermal tests for IgE to tree pollen and other environmental allergens.

Patients who have IgE antibodies that detect pollen, dust mites, dander and other allergens are sometimes treated with immunotherapy. Immunotherapy helps these patients by allowing their sensitivity to these allergens to be reduced over time. The mechanisms behind the benefits of immunotherapy have not been elucidated. There are some mechanisms that have been discussed: 1) it is thought that this therapy reduces the number of IgE antibodies; 2) It is thought that immunotherapy somehow reduces the sensitivity of IgE antibodies over time; and 3) It has been shown that other antibodies of the Immunoglobulin type G (IgG) are produced and that these may act as blocking antibodies, i.e. that they bind to the allergens so that IgE does not bind to them.

Patients who receive immunotherapy tend to have a clinical response within a few months. However, most of these patients still have identifiable IgE antibodies by RAST or Immunocap testing and they remain reactive to skin testing. This occurs not only with environmental allergens but also with insect venoms. At John's Hopkins University, patients with a history of insect venom anaphylaxis were desensitized within 6-8 weeks by receiving repetitive injections of the offending venom. Almost none of these patients reacted to challenge stings after 6-8 weeks of therapy but almost all of them still had skin test reactivity. Skin test reactivity suggests that the IgE pathway is still active and is not what is shutting off with immunotherapy. Pathways involving Toll Like Receptors and allergic neurophylaxis may be shutting down.

Certain patients who do not have IgE antibodies to allergens are still reactive in some way to these allergens. For example, patients who have been injected with high doses of allergens in the same manner in which allergy shots are given will exhibit an inflammatory response that results in redness and swelling at the shot site. This inflammatory response does not occur quickly, as would be expected from an IgE type response. It starts a few hours after the shots and generally peaks in intensity at 12-48 hours, see FIG. 6. This delayed response may be labelled under the general term “delayed type hypersensitivity.”

Delayed type hypersensitivity (DTH) has never been thought to participate in allergic rhinoconjunctivitis or allergic asthma. In order for patients to be eligible to receive allergen immunotherapy they first have to be identified as having IgE antibodies. This is the standard of care in the practice of allergy and Medicare and other insurance payers will not reimburse for immunotherapy unless IgE antibodies are first identified that are reactive to allergens that fit the pattern of symptoms the patient is experiencing.

The present invention includes the use of allergen immunotherapy for what has been termed “non-allergic” rhinoconjunctivitis, “non-allergic” asthma, delayed type hypersensitivity to environmental allergens, and for allergic neurophylaxis. Immunotherapy for non-IgE mediated allergy is administered in the same manner as for patients with IgE mediated allergic conditions except that the dose of allergens required to see improvements is often much less than that needed for patients affected by IgE responses. It can be administered by subcutaneous injections typically once or twice weekly with this interval becoming less frequent over time, with the interval eventually reach 4 weeks or even 6 weeks in some cases. For purposes of example only and not intended to be limiting, a patent may receive weekly injections, then biweekly injections, then triweekly, quadweekly, pentweekly, and then hexweekly injections, as treatment progresses. It can also be administered via the sublingual route whereby allergens are administered daily under the tongue by using a liquid dropper or spray bottle. It can also be administered under the tongue using tablets containing allergens. Sublingual immunotherapy can be administered daily or as infrequently as weekly, including administrations every other day, every third, fourth, fifth, or sixth day, etc. Patients receiving immunotherapy for non-IgE mediated immune responses do not typically require a “build-up” phase utilizing increasing doses over time. Rather, they often respond to low, starting doses that are simply continued at the same dose. In contrast, patients with IgE mediated allergies who are treated with allergy shots (subcutaneous injections) typically start at a low dose that is increased to much higher doses over several months. Patients with IgE mediated allergies who are treated with sub-lingual immunotherapy do not require a build-up phase, but the dose used is typically much higher than that required for patients with non-IgE mediated responses. The dose required for non-IgE mediated responses can be 1,000 to 1 million times lower than for IgE mediated responses.

There is evidence that delayed type hypersensitivity to pollen is involved in the development of Kawasaki's vasculitis, an inflammatory condition affecting blood vessels. It is proposed that delayed type hypersensitivity is probably involved in many inflammatory conditions, such as Crohn's disease, Ulcerative colitis, Rheumatoid Arthritis, Systemic Lupus Erythematosis, Idiopathic Pulmonary Fibrosis, Interstitial Cystitis, and other chronic inflammatory conditions.

Inflammatory bowel disease (Crohn's/Ulcerative colitis) is thought to be increasing for the same reasons that allergic conditions are increasing, that is because of the hygiene or “old friends” hypotheses. No cause/effect relationship has been suggested between allergy and inflammatory bowel disease but the similarity in theories behind their development suggests an underlying link.

Interstitial Cystitis (IC) has been found to occur concomitantly with symptoms of rhinoconjunctivitis. Histopathologic abnormalities resembling that of allergic disorders, including mast cell activation, histamine release and eosinophil infiltration are also seen with this condition. However, many patients with interstitial cystitis do not exhibit IgE responses to skin testing and therefore a definitive link between allergy and IC has not been established. In one case, a 28 year old female who was treated with immunotherapy for allergic rhinitis had improvements in her IC. Her doses of immunotherapy were administered at 1000× lower than a dose that at one point caused anaphylaxis. The applicant believes that underlying non-IgE mediated immune responses caused this patients IC, even though IgE pathways were also present and active. Her response to immunotherapy might be related to the low doses administered. IgE responses are known to be reduced most effectively at high doses with a continuously increasing dose response curve. The applicant's experience is that non-IgE mediated pathways respond to much lower doses.

The Tsimane people in South America live as our ancestors did many years ago, hunting, foraging and farming small plots of land with few comforts of modern Westernized societies. Seventy percent of these people are infected with parasites. These people do not develop Alzheimer's disease even if they have two copies of the ApoE4 gene, something that increases the risk for Alzheimer's disease in Westerners. Those with the highest degree of parasitic infection have the most protection against cognitive decline with age. This provides strong evidence that parasites protect from Alzheimer's. There is also increasing evidence that an immunologic process causes Alzheimer's. The current disclosure proposes that the Tsimane people are protected from Alzheimer's because their parasitic infections protect them from developing allergy (the “Old Friend's hypothesis”). The current disclosure proposes Delayed Type Hypersensitivity or other non-IgE mediated immune responses to environmental allergens is the cause of Alzheimer's disease and that it could be slowed or blocked with allergen immunotherapy.

The Tsimane people also have very low risk of cardiovascular disease. 85% of them can live their entire lives without any atherosclerosis in the arteries of their heart. Inflammation is thought to contribute to cardiovascular disease and inflammatory markers such as C-reactive protein have been used to assess risk of cardiovascular disease. The current disclosure proposes that DTH or other non-IgE mediated immune responses to environmental allergens may also cause or contribute to cardiovascular disease and that allergen immunotherapy could mitigate it.

In a further embodiment, the current disclosure relates to systems and methods for treating gastroesophageal reflux disease and, more particularly, a new use of immunotherapy for treating gastroesophageal reflux disease. Gastroesophageal reflux disease (GERD) or acid reflux is when stomach acid enters the esophagus. The applicant has discovered that immunotherapy can conveniently treat and reduce acid reflux. For example, patients have been treated with 400 microliters of liquid containing allergens under the tongue at varying concentrations (typically 10,000-10 million times less concentrated than stock extracts available from extract manufacturers such as Stallergenes Greer, Jubilant Hollister Stier or ALK Abello). Other patients are treated with subcutaneous injections given once or twice weekly or sometimes monthly with similar concentrations in doses of 0.1-1 milliliter.

The typical dose that is effective for most patients is 5000-10,000 times lower than stock solutions available from extract manufacturers. For example, if a patient were found to have either an IgE mediated or delayed type hypersensitivity to dust mites, pet dander and weed pollens a formulation could be produced as follows. 1 cc of stock mixed dust mite extract is mixed with 1 cc dog extract, 1 cc cat extract 0.5 cc's of ragweed and 1.5 cc's of a mixture of other weed pollens into a 5 cc vial. By mixing these extracts together they are already diluted by a factor of 5-10× compared to stock extracts. This vial is then further diluted by a factor of 10 in serial fashion (0.5 cc's added to 4.5 cc's of saline would accomplish this). The resulting vial then contains extract at 50-100× lower concentrations than stock extracts. If this dilution is again done in series two more times a vial will then contain extract at 5,000 to 10,000 times lower concentration than stock extract. Some patients with non-IgE mediated hypersensitivities may experience an increase of symptoms with immunotherapy if the dose is too high. In these patients the dose can be lowered by a factor of 10, 100 or 1000 until worsening stops and improvement is seen.

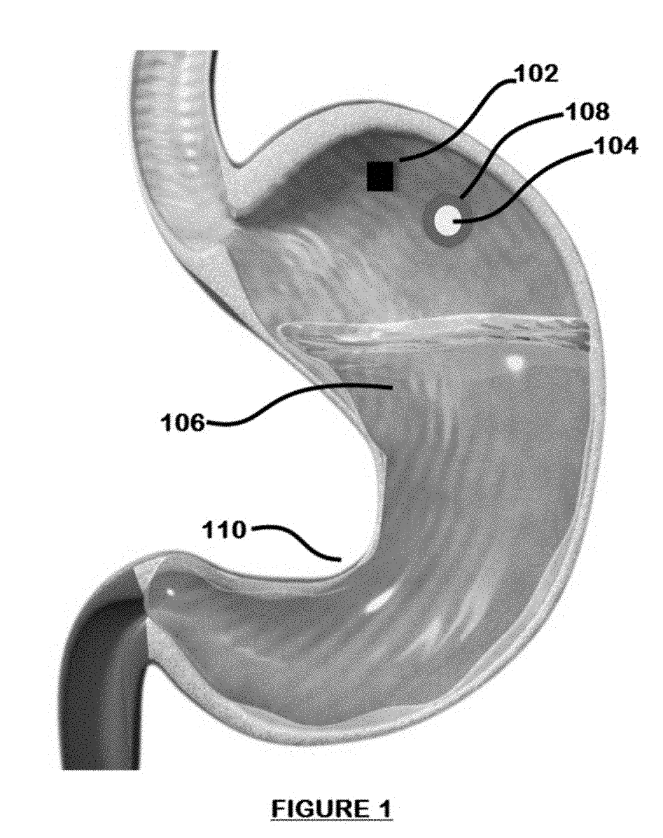

Histamine is an established mediator of mast cell activation and a known parasite killer. FIG. 1 illustrates that histamine 102 stimulates proton pump 104, that makes stomach acid 106, through activation of the H2 receptor 108 in stomach 110. This suggests that the major function of stomach acid is parasitic killing, not digestion. Acid reflux is proposed to be a defensive response against parasitic invaders. It would also be activated by false parasites, such as allergens, which express PAMPs, just as true parasites do.

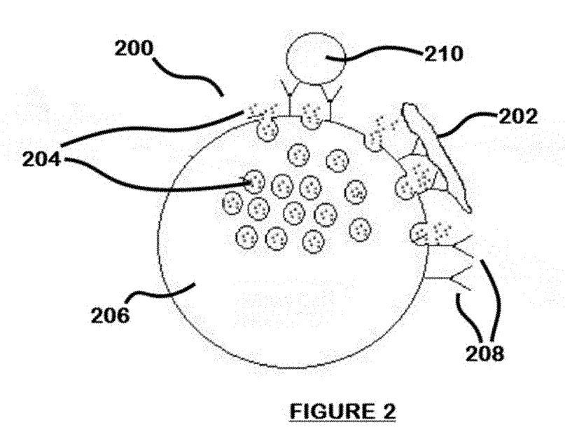

FIG. 2 shows an illustration of the classic IgE defensive response 200 against parasites 202. Histamine 204 is released by Mast Cells 206 after IgE antibodies 208 is cross-bridged by parasitic proteins. This same defensive response occurs in what has been understood to be the classic allergic response when allergic proteins, such as pollen 210, cross-bridge IgE antibodies. Histamine is identified as an important mediator of anti-parasitic and allergic responses. This supports the concept in FIG. 1 that one of the major functions of stomach acid is also parasitic killing since the acid pump (proton pump) is activated by histamine. The immune system uses Mast Cells to kill large infectious organisms, such as parasites. Histamine is an important mediator of the Mast Cell response. Thus, one of the major functions of histamine is aiding in the destruction of parasites. The allergic response and the anti-parasitic immune response are the same response. Allergy develops in societies that eradicate parasites. This causes the immune system to mistakenly identify environmental allergens as parasites because they express PAMPs similar to those on parasites.

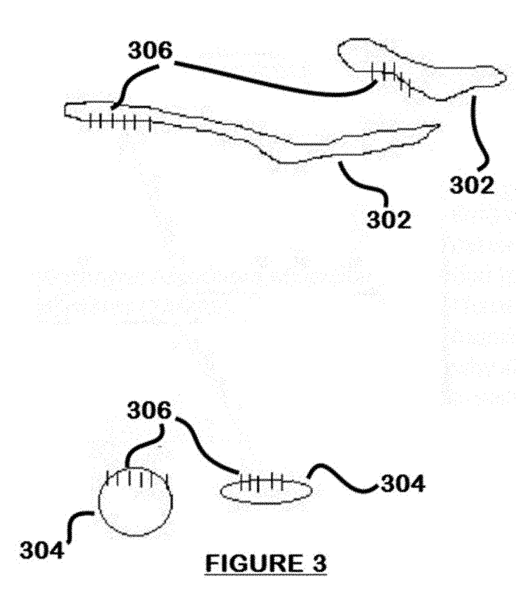

FIG. 3 shows representative images of parasites and allergens. FIG. 3 illustrates that both parasites 302 and allergens 304 express molecular patterns 306 that are similar and are referred to as pathogen associated molecular patterns (PAMPs). Both parasites and allergens express protein patterns recognized by the immune system that trigger activation of immune responses. These protein patterns are called Pathogen Associated Molecular Patterns. This may be why allergens are easily mistaken for parasites in societies that lack parasites.

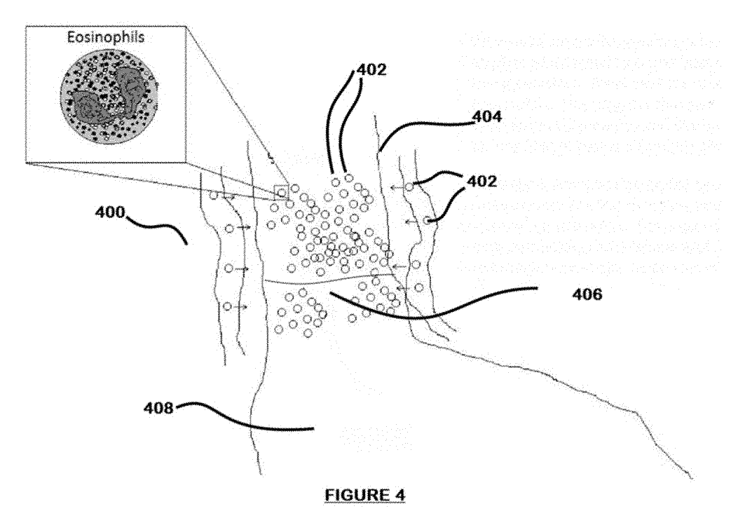

FIG. 4 shows a representative image of acid reflux 400. It identifies a unique type of white blood cell, eosinophils 402, infiltrating the esophagus 404 in response to acid 406 from stomach 408. Since eosinophils 402 specialize in killing parasites this suggests that acid 406 has parasitic killing activity and acid 406 has evolved to be a surrogate signal for the presence of parasites.

Because allergens express PAMPs, they could also stimulate acid reflux through pathways not involving IgE. Allergen immunotherapy may reduce acid reflux. When acid enters the esophagus (acid reflux) it attracts white blood cells, Eosinophiles, that specialize in killing parasites. This suggests that acid itself is intended to kill parasites and that reflux could be intended to have a protective effect. If allergens are falsely perceived as parasites, as they express PAMPs, then they could be stimulating acid reflux. This suggests that Immunotherapy could reduce acid reflux by reducing immunologic responses to allergens.

Evidence in the medical literature suggests a long-standing relationship between stomach acid and parasites since patients with GERD often have parasitic killing cells, called eosinophils, infiltrating the esophagus, see FIG. 4. This suggests that acid has been killing parasites for so long that the body evolved to utilize stomach acid as a surrogate signal for the presence of parasites. As a result, the applicant views acid reflux to be the body's defensive response against perceived parasitic invaders, which in many instances is activated by a false alarm triggered by “false” parasites—i.e., allergens. The current disclosure proposes that because parasites and allergens express the same or similar pathogen associated molecular patterns (PAMPS), and because these PAMPS are recognizable by the immune system, allergens can be mistaken for parasites by the human body—and as a corollary, immunotherapy can treat and reduce acid reflux by reducing the immunologic responses to allergens.

To reiterate, since stomach acid's function is not just digestion, but more importantly an immuno-protective response against parasites, allergens are false parasites causing acid reflux as a defensive response, a response immunotherapy can correct, thereby reducing GERD.

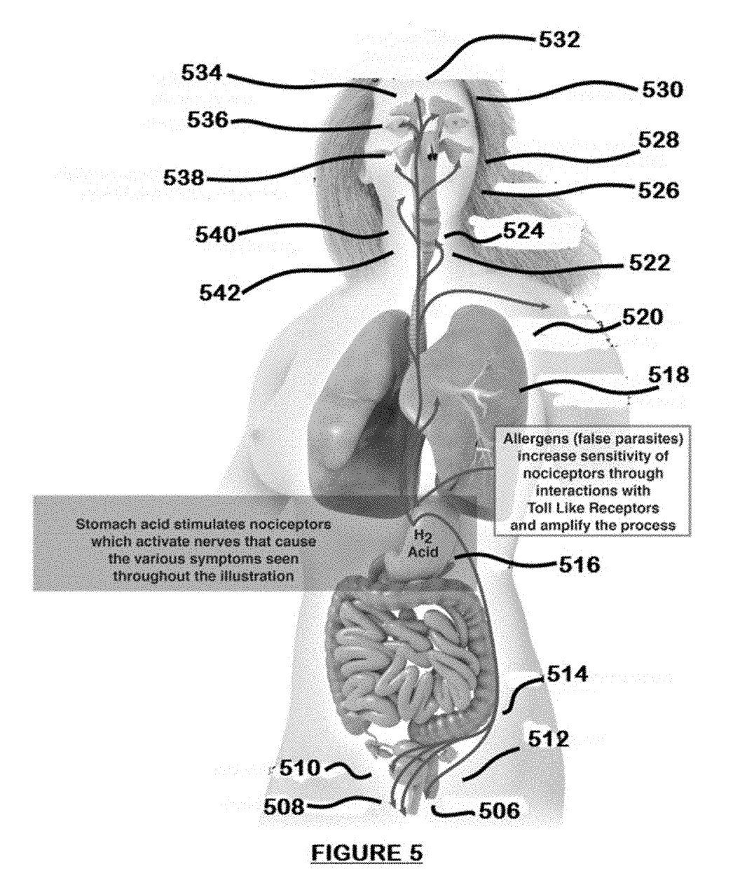

FIG. 5 identifies neurophylaxis. Neurophylaxis identifies that the pathways that have been traditionally called “atypical acid reflux” and some of the pathways of allergy are the same pathways and involve stomach acid, nerves, and amplification through Toll Like Receptor activity. It also identifies a more diffuse immune response than that previously recognized as being associated with reflux or allergy. It further identifies that atypical acid reflux is involved in the development of diffuse symptoms not limited to cough and hoarse voice but affecting every orifice of the body and the skin. It identifies that allergens stimulate these pathways. This figure reveals a proposed pathway of immunology that the applicant has termed neurophylaxis (a fending-off response mediated by the nervous system). It ties allergy to acid reflux and both to the development of a large group of diffuse, previously disparate symptoms.

FIG. 5 illustrates that atypical acid reflux 500 occurs when stomach acid 502 stimulates nociceptors (sensory receptors) 504 which in turn activate nerves, not shown, that cause the various symptoms illustrated in FIG. 5. Allergens, acting as false parasites, increase sensitivity of nociceptors through interactions with Toll Like Receptors and amplify the process. These symptoms, which may appear in various combinations, may include, but are not limited to, vulvodynia 506, pollakiuria 508, interstitial cystitis 510, puritus ani 512, diarrhea/irritable bowel 514, nausea 516, shortness of breath, chest tightness 518, pruritus (neurodermatitis and/or atopic dermatitis) 520, throat clearing/hoarse voice 522, glossodynia 524, ear pressure, tinnitus, vertigo, excess ear wax 526, frontal headache 530, post nasal drip/sinus pressure (recurring “sinus infection”) 532, eyelid edema 532, allergic shiners 534, itchy/watery eyes 536, sneezing/epistaxis/nasal congestion/loss of smell/taste, rhinorrhea 538, sore throat/itchy pharynx 540, and cough 542.

The claimed invention differs from what currently exists. The current treatments for GERD involve medications, life-style modifications, and fundoplication surgery. The proposed method corrects the source of the problem, which is the splashing of stomach acid up the esophagus to protect from pathogens, especially parasites. Since allergens are false parasites, exposure to them activates GERD, and correcting allergic reactivity by the use of immunotherapy has a calming effect on GERD.

The current paradigm of allergy suggests that an allergic response is stimulated through activation of Immunoglobulin type E (IgE) antibodies. The current disclosure proposes that other pathways are also involved that do not require stimulation of IgE. Allergens are likely also stimulating Toll Like Receptors (TLRs), the receptors that recognize PAMPS. Immunotherapy for GERD is understood by the inventor to be more effective if the therapy utilizes allergens that are detected by IgE and by other pathways. IgE mediated allergies can be detected within minutes in patients by traditional skin prick and intradermal tests. Alternatively, allergen specific IgE antibodies can be measured by blood tests such as radioallergosorbent (RAST) testing or ImmunoCap testing. Detection of other allergic sensitivities, such as DTH, can be achieved either by injecting allergens subcutaneously at a dose approximately 100-1000× times stronger than intradermal tests and at volumes of 0.25-1 cc or by injecting allergens intradermally (0.02-0.05 ml's) at 10×-100× higher doses than traditional intradermal tests that are used for evidence of IgE activation. It should be understood that patients do not have to exhibit IgE responses or DTH responses to have a response to immunotherapy. It is assumed that patients can have a hypersensitivity response not involving IgE or DTH that involves Toll Like Receptor activation or other unknown immunologic pathways that stimulate GERD. DTH testing may simply be another way of identifying that the immune system is attacking a specific allergen. It is assumed that any form of attack may be accompanied by other forms of attack. Even patients who have IgE mediated allergy can also have activation of other underlying pathways involving DTH and/or TLR activation. For example, a patient with allergic rhinitis and positive IgE tests may also have Crohn's disease. The applicant's experience is that patients in this setting who are treated with traditional immunotherapy will have improvements in symptoms of rhinitis, but not Crohn's. In fact, their Crohn's disease may worsen. However, if these patients are treated with much lower doses (1000-1 million times lower) of immunotherapy they can have improvements in Crohn's symptoms. This correlates with what was seen in the 28 year old patient with interstitial cystitis.

To avoid anaphylaxis, tests for DTH are only performed in patients who do not have IgE antibodies that recognize the allergens being used. Since these new tests are not activating an IgE/Mast Cell response they do not elicit the classic wheal and flare response elicited by traditional skin tests. They elicit a delayed type hypersensitivity response that causes induration, erythema and swelling starting 6-12 hours after injection and peaking at 24-72 hours, see FIG. 6. As can be seen, there is a need for a new use of immunotherapy for treating gastroesophageal reflux disease.

Broadly, an embodiment of the present invention provides a new use of immunotherapy for treating non-IgE mediated environmental hypersensitivities causing gastroesophageal reflux disease, atypical acid reflux, non-allergic rhinitis, non-allergic asthma, neurophylaxis, inflammatory bowel disease and other inflammatory conditions. It has been unexpectedly discovered according to the present invention, that the use of immunotherapy has beneficial results for treating these conditions. Accordingly, the object of the present invention, generally stated, is the provision of improved GERD treatment through to the incorporation of an effective immunotherapy regiment.

Referring now to FIGS. 1 through 5 in reference to GERD and atypical manifestations of GERD, which the applicant refers to as neurophylaxis, the applicant of the present invention postulates the following: since histamine, a known parasite killer, stimulates stomach acid through activation of the histamine type 2 receptor, and because stomach acid attracts eosinophils, the major function of stomach acid is not digestion but rather the body's parasitic defensive response, which in many instances is activated by “false” parasites—i.e., allergens. The applicant theorizes that because parasites and allergens express the same or similar pathogen associated molecular patterns (PAMPS), and because these PAMPS are recognizable by TLRs of the immune system, allergens can be mistaken for parasites by the human body, and so as a corollary immunotherapy can treat and reduce acid reflux and neurophylaxis by reducing the immunologic responses to allergens.

A method of using the present invention includes the following. Immunotherapy in the form of allergy shots, liquid sublingual drops or tablets containing allergens may be used to treat acid reflux, atypical acid reflux (neurophylaxis), non-allergic rhinoconjunctivitis, non-allergic asthma and inflammatory conditions such as Crohn's disease, Ulcerative Colitis, Alzheimer's disease, Coronary Artery disease and others. For example, allergy extracts such as those sold by Stallergenes Greer, Jubilant Hollister Stier or ALK Abello or tablets such as Grastek™ a tablet for grass allergy or Ragwitek™ a tablet for ragweed allergy, may be taken to reduce or alleviate the symptoms of GERD. For subcutaneous immunotherapy, vials are filled with 0.5-1 cc of various allergens from stock concentrations to fill a 5 cc vial. No diluent is added and the vial contains only stock extract. For example, one vial may contain 1 cc mixed dust mite, 1 cc dog extract, 1 cc cat extract, 0.75 cc's Ragweed and 1.25 cc's of stock weed mix. This vial is then diluted by a factor of 1000 to 100,000 depending on the severity of symptoms and/or the size of the response of DTH testing. 0.5 cc's of the dilute concentration is administered subcutaneously once or twice weekly with the interval spaced out over time as the patient improves. For sublingual immunotherapy a vial containing only stock extracts is formulated and diluted by a factor of 100 to 10,000 depending on severity of symptoms and the size of the reaction to DTH tests. The diluted solution is placed into a spray bottle available from Stallergenes Greer and other companies designed to administer a precise volume of solution. The applicant's vials administer 200 ul per spray and the patients are instructed to squirt 2 sprays under the tongue daily. Over time, some patients can reduce the dosing schedule to every other day, every third day and eventually every fourth day.

### EXAMPLE 1

Mr. X, a 67-year-old male, presented to the Allergy & Asthma Center of Hilton Head in 2016 complaining of frequent symptoms of post nasal drip, cough, throat clearing, and recurring sinus pressure, which he presumed represented infections. Traditional skin prick and intradermal tests were performed and showed significant reactivity to cats and dogs but to nothing else. The patient noticed increasing symptoms around pets but was surprised that he did not show reactivity to pollen since his symptoms tended to vary with the seasons as well. Despite the fact that the patient lived with a cat, immunotherapy was not initiated. The patient was treated with medications unsuccessfully for several months. Eventually immunotherapy was initiated and over time the patient noticed some improvement in his regular symptoms with a significant reduction in his symptoms around pets. His seasonal spring and fall symptoms continued. A delayed type hypersensitivity test was administered during the spring tree pollen season using pollens and mold. Specifically, 0.5 cc's of a tree pollen mix was administered subcutaneously into the upper arm and 0.5 cc's of grass and weed pollen mixtures into the other arm at a concentration of 1/10th that of stock extract available from Stallergenes Greer and Jubilant Hollister Stier. The patient had significant swelling in the areas where both shots were administered. The swelling began at 6 hours and peaked at 36 hours. The swelling primarily involved erythema and induration measuring approximately 10×15 cm each. Sublingual immunotherapy was initiated using low doses of pollen at a concentration of 1/10,000th of stock allergens. The patient administered 2 sprays containing 200 ul of solution under the tongue daily. He was advised to keep the liquid under the tongue for 15-30 seconds and then to swallow it. Within weeks the patients seasonal sinus pressure, post nasal drip and cough resolved. The therapy was continued and the patient also noticed a lack of symptoms later that fall when he normally would experience a severe sinus “infection” but remained symptom free.

### EXAMPLE 2

Mrs. X is a 71 year old female who presented complaining of chronic itching in the nose and throat associated with runny nose, sneezing, watery eyes and cough. Her symptoms have been present on a perennial basis for more than three years affecting her daily. Traditional prick and intradermal allergy skin tests were negative to a panel of 64 different allergens. Four allergy shots containing high doses mite/pet/weed mixes, grass mix, tree mix and mold mix were administered in different positions on both arms. The patient returned two days later for re-evaluation. The mold shot had no swelling. The other three shots all swelled to approximately 13-20 cm in diameter. The patient was started on sublingual immunotherapy containing dust mite, pet danders, weed mix, grass mix, tree mix at concentrations 1000 times less concentrated than stock extracts available from Stallergenes Greer and Hollister Stier. She was advised to administer 2 sprays under the tongue daily. Each spray contained 200 ul of the dilute solution of allergens. She returned two weeks later to report possible improvements and by 10 weeks her improvements were obvious with an estimated 75% improvement in all symptoms. She reported that she had not felt better in three years.

### EXAMPLE 3

Mrs. Y is a 64 year old female who presented complaining of nasal congestion, sinus pressure and post nasal drip. Screening for other symptoms revealed daily headaches, fatigue and a diagnosis of irritable bowel syndrome. Her irritable bowel manifested as frequent diarrhea often with several days of constipation alternating back and forth. She had traditional allergy skin prick and intradermal tests for 64 allergens with no evidence of reactivity. Four allergy shots containing high doses of mite/pet danders/weed mix, grass pollen mix, tree pollen mix, and mold mix were administered in varying positions on the upper arm. Within 6 hours there was significant swelling of the mite/pet/weed shot which peeked at 36 hours. The grass and tree shot showed minimal swelling and the mold shot did not swell at all. She had indigestion, moderate headache and post nasal drip 6 hours after the test lasting approximately 24 hours. Sublingual immunotherapy was initiated containing small amounts of mite/pet danders/weed mix at 10,000× lower concentration than stock extracts. Within three weeks the patient reported dramatic improvements in all symptoms including headaches, sinus pressure, nasal congestion, post nasal drip, fatigue and symptoms of IBS. She was having normal bowel movements for the first time in years. These benefits were sustained on daily drops (400 ul daily dose) at 10 weeks and at 20 weeks.

### EXAMPLE 4

Mrs. SY is a 71 year old female who presented complaining of chronic cough. The cough was most active during spring and fall months but was present to some degree on a perennial basis over the past 10 years. She was thought to have acid reflux but omeprazole 20 mg twice daily did not suppress her cough. Upon evaluation SY was found to have no IgE mediated allergies by traditional skin prick and intradermal testing. Her dose of omeprazole was raised to 40 mg twice daily and her cough improved by 80%. After three months of therapy the patient became fearful of bone density loss on omeprazole after being warned of this side effect by her primary care physician. She returned to seek other options. She was injected with four shots to look for delayed type hypersensitivity (DTH). One contained dust mites, pet danders and weed pollen. A second contained a mixture of grass pollens and minor tree pollens. A third contained major tree pollens and a fourth contained a mixture of mold spores. These specific combinations were chosen because allergy shots in the applicant's clinic are often mixed in this way. The doses of the extracts were each at a concentration of 10-20% the strength of stock extract available from Stallergene's Greer or Jubilant Hollister Stier. For example the mite/pet/weed shot contained 1 cc stock mixed mite, 1 cc stock dog extract, 1 cc stock cat extract, 0.75 cc's stock Ragweed Mix and 1.25 cc's stock weed mix. This type of vial would be known as a 1:1 maintenance immunotherapy vial since it is often the final concentration achieved in immunotherapy when given to IgE reactive patients. A dose of 0.5 cc's of each of these mixtures was injected subcutaneously into different sites in the upper arm. She exhibited significant redness and swelling at the shot sites measuring approximately 20-30 cm in diameter. Her Prilosec was stopped and she was started on subcutaneous immunotherapy. Four shots of immunotherapy were prepared by taking the same vials used for the DTH test and diluting them by a factor of 100,000 (called 1:100,000). A starting dose of 0.05 cc's was administered of each shot and the dose was slowly increased over time (0.05, 0.1, 0.2, 0.3, 0.4, 0.5 . . . then 1:10,000 0.05, 0.1, 0.2, 0.3, etc., as is typical of immunotherapy when given for IgE mediated allergies). Within three weeks of starting twice weekly injections (6 doses into therapy) she reported complete resolution of her cough. Her doses were increased towards the maintenance (1:1) concentration over time but when the patient reached the 1:100 concentration 0.1 cc's she noticed that her shots were stimulating cough approximately 2 hours after the shots. When she reached 1:10 0.2 cc's her persistent cough returned. Her doses were reduced to 1:1,000 0.5 cc's and her cough subsided again within weeks. She was maintained with weekly injections of this dose and after 1 year she reported that she had been 99% symptom free of cough over the past 8 months. The most interesting aspect of this case is that SY responded both to high doses of omeprazole and to immunotherapy (low dose). This suggests a relationship between allergy and acid reflux. This has not yet been established probably because we have only been using IgE type tests to identify allergy. If other pathways of environmental hypersensitivities are considered, a strong relationship between allergy and acid reflux can be elucidated.

### EXAMPLE 5

Mrs. CM is a 74 year old female with a history of sinus pressure, nasal congestion, post nasal drip, cough and watery eyes occurring only during spring and fall months for many years. Screening for other symptoms revealed urinary urgency, which also occurred typically only during the spring and fall. She noted that her spring symptoms correlated with seeing yellow pollen on the cars and on the ground. She had traditional IgE skin prick and intra-dermal tests which were non-reactive. Because her symptoms were so suggestive of allergy she also had a blood/RAST test for environmental allergies which was also negative. Four shots (mite/pet/weed, grass/minor tree, tree, and mold) of 0.5 cc's were injected at a 1:1 concentration (typical maintenance dose allergy shots) at various sites in the upper arms. Swelling occurred, including induration and erythema measuring approximately 15-30 cm in diameter to the mite/pet/weed and major tree pollen shots. Immunotherapy was initiated starting at a concentration of 1:1000 0.5 cc and this dose was administered twice weekly for two months and weekly thereafter. The therapy was started during the summer months when the patient was asymptomatic. She expected her symptoms to recur during the fall months but they did not. In similar fashion her symptoms were absent during the spring for the first time in more than 10 years.

### EXAMPLE 6

Mrs. PC, a 28 year old female presented complaining that she must have a terrible food allergy since her Crohn's disease had been flaring out of control for three years and her gastroenterologist was unable to gain control of her symptoms. Skin testing for IgE mediated food allergies was negative. After a lengthy discussion the patient agreed to a work-up for environmental allergies. IgE skin tests were negative. DTH tests were performed by administering 4 injections of 1:1 maintenance doses of allergy shots (containing mite/pet/weed, grass pollen mix, tree pollen mix, and mold mix). The patient was asked to return in 2 days for re-evaluation. The patient missed her follow-up appointment. Three weeks later she reported back to report that she had been hospitalized with rectal bleeding, abdominal cramping, and diarrhea indicative of a severe flare of her Crohn's disease. It was the first time she had been hospitalized over-night for Crohn's. She explained that the symptoms started the evening of her DTH test and by 24 hours after the test she was in the Emergency Room. At that time her arms were swollen severely (20-30 cm diameter swelling) at all 4 shot sites of the DTH tests. She was admitted to the hospital for almost a week where she required a blood transfusion. She wondered if the test stimulated her symptoms. She was started cautiously on Immunotherapy at doses of 1:100,000^{th} of the doses used for the DTH test at volumes of 0.05 ml's for each of the 4 shots. She had a mild flare of her symptoms three hours after the first set of shots. Her dose was reduced by a factor of 10 and twice weekly shots were continued from this lower concentration. Her doses were initially increased from 0.05 to 0.1, 0.2, then 0.3 where she was maintained (this dose is approximately 10 million times lower than stock concentrations available from Stallergenes Greer or Jubilant Hollister Stier). Within three weeks her symptoms were 90% improved. This improvement persisted beyond 20 weeks of treatment.

Applicant perceives there to be many immune responses that could be occurring in response to environmental allergens. Applicant's research shows that an immune response often starts with Toll Like Receptor activation and this type of immune response contributes to what is herein called allergic neurophylaxis (see FIG. 6) by stimulating autonomic nervous system activity that is working in an immunologic fashion by creating flushing, expelling phylactic responses. Later, the body may progress to utilize white blood cells which can infiltrate an area and use various methods of attack. This is a typical delayed type hypersensitivity response and this response may not involve Toll Like Receptors. Later an immune response can activate an antibody response utilizing IgE in the case of allergy. To better understand the current disclosure, the following helps contextualize allergic reactions. Soldiers, guns and bullets are like a TLR response. Tanks, bombs and jets are like a WBC/DTH response and Missiles/Nuclear weapons are like an IgE antibody response. In the same way one does not have to fire nuclear weapons to be at war, one does not have to have IgE to be fighting pollen. Patients who have IgE responses often presumably have DTH responses and TLR responses, but, critically, DTH and TLR responses are important and cannot be identified purely by IgE tests. IgE and DTH tests may be considered as ways to establish that a war is occurring between the body and pollen (or other allergens). Many wars/allergen conflicts will be missed by looking only for IgE. However, some patients (although very rare) will have negative IgE responses and negative DTH responses but still respond to immunotherapy because they have an underlying TLR response for which currently there is no assay or way to look for it. The current disclosure believes neurophylaxis is primarily mediated by TLR responses but Applicant uses DTH tests to identify which allergens the body is fighting. Applicant assumes that inflammatory conditions, such as Crohn's disease are being mediated by DTH/white blood cell responses.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A method for treating delayed type hypersensitivity to allergens comprising:
providing immunotherapy to a mammal exhibiting allergy symptoms via administering allergens to a mammal via a delivery system; and
wherein the mammal has a negative result for IgE antibodies.

2. The method of claim 1, wherein the delivery system for the allergens comprises subcutaneous injections, liquid sublingual drops, tablets, or combinations of the above.

3. The method of claim 2, wherein subcutaneous injections are delivered over increasing time intervals.

4. The method of claim 1, wherein the dose of allergen remains substantially constant during the administration of the allergen.

5. The method of claim 2, wherein the dose of allergen administered via liquid sublingual drops is 1,000 to 1,000,000 times lower than an allergen dose for IgE mediated allergy responses.

6. The method of claim 1, used to treat acid reflux, atypical acid reflux (neurophylaxis), non-allergic rhinoconjunctivitis, non-allergic asthma and inflammatory conditions such as Crohn's disease, Ulcerative Colitis, Alzheimer's disease, and/or Coronary Artery disease.

7. The method of claim 1, wherein the allergen is selected from the list comprising dust mite, dog extract, cat extract, Ragweed extract, stock weed pollen mix, mold spore mix, tree pollen mix, or combinations of the above.

8. A method for treating allergic reactions comprising;
treating allergic reactions in a mammal wherein at least one allergen causing an allergic reaction in the mammal possesses a pathogen associated molecular pattern similar to a parasitic pathogen associated molecular pattern; and
providing immunotherapy to the mammal via administering allergens to the mammal via a delivery system.

9. The method of claim 8, wherein the delivery system for the allergen comprises subcutaneous injections, liquid sublingual drops, tablets, or combinations of the above.

10. The method of claim 9, wherein subcutaneous injections are delivered over increasing time intervals.

11. The method of claim 8, wherein a dose of allergen provided by the delivery system remains substantially constant during the administration of the allergen.

12. The method of claim 9, wherein a dose of allergen administered via liquid sublingual drops is 1,000 to 1,000,000 times lower than an allergen dose for IgE mediated allergy responses.

13. The method of claim 8, used to treat acid reflux, atypical acid reflux (neurophylaxis), non-allergic rhinoconjunctivitis, non-allergic asthma and inflammatory conditions such as Crohn's disease, Ulcerative Colitis, Alzheimer's disease, and/or Coronary Artery disease.

14. The method of claim 8, wherein the allergen is selected from the list comprising
dust mite, dog extract, cat extract, Ragweed extract, stock weed pollen mix, mold spore mix, tree pollen mix, or combinations of the above.

15. A method for treating gastroesophageal reflux disease in a mammal comprising:
administering immunotherapy to a mammal, wherein the immunotherapy is administered via a delivery system;
wherein the delivery system contains allergens.

16. The method of claim 15, wherein the delivery system for the allergen comprises subcutaneous injections, liquid sublingual drops, tablets, or combinations of the above.

17. The method of claim 16, wherein subcutaneous injections are delivered over increasing time intervals.

18. The method of claim 15, wherein a dose of allergen provided by the delivery system remains substantially constant during the administration of the allergen.

19. The method of claim 9, wherein a dose of allergen administered via liquid sublingual drops is 1,000 to 1,000,000 times lower than an allergen dose for IgE mediated allergy responses.

20. The method of claim 15, wherein the allergen is selected from the list comprising Dermatophagoides farina, Dermatophagoides pteronyssinus, Dust mite mix, cockroach, dog extract, cat extract, horse extract, Ragweed extract, cocklebur, English plaintain, marsh elder, lamb's quarter, pigweed, mugwort, Japanese hop, Bermuda, timothy, meadow fescue, June, Orchard, Red Top, Perennial Rye, Bahia, Sweet Vernal, Brome, Johnson, White ash, privet, White birch, Alder, Oak, Beech, Cottonwood, Black willow, poplar, pecan, black walnut, shagbar hickory, maple, box elder, elm, sweetgum, Sycamor, cedar, bayberry, mulberry, Alternaria, Aspergillus, Helminthosporium, Hormodendrum, Penicillium, Fusarium, Mucor, Phoma, Candida, Epicoccum, Rhizopus, Pullularia, Curvularia, Stemphyllium and Chaetonium or any combinations of the above.

21. A delayed type hypersensitivity test comprising:
injecting a mammal with a reduced concentration of allergen stock;
monitoring the mammal for signs of a reaction to the allergen stock;
wherein a reaction to the allergen stock is delayed in that no immediate reaction to the allergen stock occurs but instead an initial reaction begins hours after the injection and peaks hours after the initial reaction.
